(19) United States
(12) Patent Application Publication
(10) Pub. No.: US 2010/0318012 A1
(43) Pub. Date: Dec. 16, 2010

(54) BACTERIOSTATIC TEXTILE BASED ON POLYAMIDE 11

(75) Inventor: Sophie CHHUN, Limeil-Brevannes (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Colombes (FR)

(21) Appl. No.: 12/815,760

(22) Filed: Jun. 15, 2010

(30) Foreign Application Priority Data
Jun. 16, 2009 (FR) 09.54018

(51) Int. Cl. A61F 13/00; B32B 5/02; B32B 3/26; D03D 15/00; D04H 13/00; D04B 1/00; D04B 21/00; D04H 1/08

(52) U.S. Cl. 602/43; 428/323; 428/317.9; 442/181; 442/327; 442/304; 442/324

(57) ABSTRACT

The present invention relates to a bacteriostatic textile material of polyamide 11 where said polyamide 11 has an inherent viscosity of from 0.5 to 1.7 and contains no impurities with a diameter greater than 5 μm. The bacteriostatic textile material is useful in the fields of medicine, hygiene, baggage, clothing manufacture, clothing, household equipment and goods, upholstery, carpets, automobiles, industry, notably industrial filtration, agriculture and/or building construction.

### FIELD OF THE INVENTION

The subject of the present invention is a bacteriostatic textile comprising synthetic fibers made of a thermoplastic polymer.

In the following description of the invention:
“textile material” or “textile” is understood to mean any material made of fibers or filaments as well as any material forming a porous membrane characterized by a length/thickness ratio of at least 300;
“fiber” is understood to mean any synthetic or natural material, characterized by a length/diameter ratio of at least 300;
“filament” is understood to mean any fiber of infinite length.

Among textiles, the following are notably found: fiber mats (dressings, filters, felt), meshes (dressings), threads (sewing threads, knitting threads, threads for weaving), knitwear (rectilinear, circular, “fully-fashioned”), fabrics (traditional fabric, Jacquard fabric, multiple fabric, double faced fabric, multiaxial fabric, 2D and a half fabric, 3D fabric) and many others.

Textiles are classified in three categories according to their activity toward bacteria:
a textile is said to be “neutral” when it exhibits no particular activity, bacteria developing normally on this textile;
a textile is said to be “bacteriostatic” if bacteria develop very slowly but do not die on this textile;
a textile is said to be “bacteriocidal” if bacteria die in contact with the textile.

### PRIOR ART

Synthetic fibers made of a thermoplastic polymer that are currently used for producing textiles with a bacteriostatic effect are intrinsically neutral. It is therefore necessary to modify these fibers or the textiles obtained from these fibers so that they acquire activity toward bacteria. The methods used consist either of carrying out a surface chemical treatment of the textile or of adding an additive that is active toward bacteria to the fiber matrix. Antibacterial agents currently used are zinc sulfide ZnS, silver salts, silver ions, sulfuric acid salts such as Al₂(SO₄)₃, ZnO, KAl(SO₄)₂, ZnSO₄, quaternary ammonium salts, triclosan, chitosan, etc.

These current techniques have several disadvantages. They require at least one supplementary production step for treating the surface and/or for adding an antibacterial additive to the mass. Moreover, these techniques may modify the flexibility and the touch of the textile. Finally, most of the agents that are active toward bacteria are harmful for the environment (silver salts, zinc salts).

In addition, consumers are increasingly attracted to textile products coming from synthetic methods that are not dependent on raw materials of fossil origin, but rather use raw materials of renewable or bio-resourced origin that have a reduced impact on the environment and that also have the reputation of being compatible with the skin.

The object of the present invention is therefore to provide bacteriostatic textile materials produced by a simple method, having the least number of steps, that does not adversely affect the flexibility or softness of the textile and which, instead of using agents that are harmful for the environment, rather uses bio-resourced raw materials.

Surprisingly, the Applicant has shown that the use of a particular polyamide 11 makes it possible to produce such bacteriostatic textile materials.

### SUMMARY OF THE INVENTION

As a preamble, it is to be stated that the expressions “comprised between” and “extending from”, that are used in the rest of this description, should be understood as meaning that the limits mentioned are included.

The object of the present invention is therefore the use of polyamide 11 for producing a bacteriostatic textile material, said polyamide 11 having an inherent viscosity comprised within the range extending from 0.5 to 1.7 and containing no impurities with a diameter greater than 5 μm.

Polyamide 11 has an inherent viscosity advantageously comprised between 0.5 and 1.5, preferably between 0.8 and 1.2 and more preferably substantially equal to 1.

Advantageously, said material is in the form of a porous membrane, a woven textile or a non-woven textile.

Advantageously, said material comprises fibers and/or filaments and/or particles based on said polyamide 11.

Advantageously, said fibers and/or filaments have a smooth surface.

The subject of the present invention is also a bacteriostatic textile which comprises polyamide 11 with an inherent viscosity comprised within the range extending from 0.5 to 1.7 and said polyamide 11 contains no impurities with a diameter greater than 5 μm.

This polyamide 11 has an inherent viscosity advantageously comprised between 0.5 and 1.5, preferably between 0.8 and 1.2 and more preferably equal to 1.

Advantageously, the content by weight of polyamide 11 represents at least 50%, preferably at least 80% of the total weight of the textile.

Advantageously, said polyamide 11 is mixed with at least one filler and/or at least one pigment and/or at least one additive.

Advantageously, said textile does not contain any additive that is active toward bacteria or any surface treatment that is active toward bacteria other than polyamide 11.

Advantageously, said textile comprises synthetic fibers obtained from bio-resourced raw materials.

Advantageously, said textile additionally contains natural fibers such as cotton, wool and/or silk, artificial fibers produced from natural raw materials, inorganic fibers such as carbon, glass, silica and/or magnesium fibers, and/or synthetic fibers other than PA 11 fibers.

Advantageously, the textile according to the invention is produced solely from bio-resourced raw materials.

Advantageously, the textile according to the invention constitutes a felt, a filter, a film, a gauze, a cloth, a dressing, a layer, a woven fabric, a knitted fabric, an article of clothing, a garment, an article of bedding, an article of upholstery, a curtain, an automobile interior covering, a functional technical textile, a geotextile and/or un agrotextile.

The object of the present invention is also the use of a textile as previously defined in the fields of medicine, hygiene, baggage, clothing manufacture, clothing, household equipment and goods, upholstery, carpets, automobiles, industry, notably industrial filtration, agriculture and/or building construction.

### DETAILED DESCRIPTION OF THE INVENTION

The object of the present invention is therefore the use of polyamide 11 for producing a bacteriostatic textile material, said polyamide 11 having an inherent viscosity comprised within the range extending from 0.5 to 1.7, advantageously from 0.5 to 1.5, and preferably from 0.8 to 1.2 and more preferably substantially equal to 1. Moreover, this polyamide 11 contains no impurities with a diameter greater than 5 μm.

Polyamide 11 (abbreviated to “PA11”) is produced from raw materials of vegetable origin. Vegetable materials have the advantage of being able to be cultivated in a large quantity according to demand, over the majority of the world and to be bio-resourced. A bio-resourced raw material is a natural animal or vegetable resource, of which the stock may be reconstituted over a short period on the human scale. It is necessary in particular for this stock to be able to renew itself as quickly as it is consumed.

PA11 is produced by the Arkema Company. A range of products based on PA 11 exist under the trade name Rilsan® 11 or Rilsan® B. The base raw material of PA11 is castor oil, extracted from the plant of the same name (common castor oil plant), from castor oil seeds. PA11 is obtained by polycondensation of amino-11-undecanoic acid. Conventionally, PA11 powders are obtained by grinding a PA11 prepolymer.

PA11 according to the invention has an inherent viscosity comprised within the range extending from 0.5 to 1.7, advantageously from 0.5 to 1.5, preferably from 0.8 to 1.2 and more preferably substantially equal to 1. Outside these inherent viscosity ranges, it may be difficult to employ polyamide 11 for producing textiles.

In the present description, all the viscosities of PA11 are measured according to the ARKEMA method: the inherent viscosity is measured at a polyamide concentration of 0.5% by weight dissolved in metacresol based on the total weight of the solution at 20° C., by means of an Ubbelohde viscometer.

The PA11 used in the present invention contains no impurities with a diameter greater than 5 μm. In point of fact, impurities present in PA11 currently produced prevent fibers or filaments from being obtained since the PA11 material breaks at these impurities instead of forming a continuous strand. The usual method for producing PA11 should therefore be modified so as to extract these impurities from PA11, for example with the aid of a suitable sieve or filter.

Advantageously, said polyamide 11 is mixed with at least one filler and/or at least one pigment and/or at least one additive.

The polymeric matrix of PA11 may notably contain additives, preferably with a diameter not exceeding 5 μm, for the same reason as that referred to previously. Said additives may for example be reinforcing fillers, fire retardants, UV protective agents, UV stabilizers, heat stabilizers, pigments, lubricants, antioxidant agents, agents for improving fluidity, flow-improving agents, film-forming agents, film-forming auxiliaries, gums, semi-crystalline polymers, preservatives and mixtures thereof. Any other type of additive in the textile field may of course also be considered.

Advantageously, said textile does not contain an additive active toward bacteria, or a surface treatment that is active toward bacteria other than polyamide 11. In point of fact, the use of PA11 according to the invention is sufficient to provide bacteriostatic properties to the textile of which it is comprised.

The present invention notably relates to the use of PA11 for producing textile materials, such as threads, fibers, filaments, films, membranes, porous membranes, woven textiles and non-woven textiles. The present invention also relates to the production of and use of PA11 particles, melted so that they can adhere to the surface of textile materials in a durable manner (wash-resistance).

Said PA11 or compositions with a thermoplastic matrix based on said PA11 may be formed of a textile material directly after polymerization, without intermediate solidification and reinforcing steps. This PA11 or these compositions may also be put into the form of granules, intended to undergo refusion so as to be put subsequently into their final form, for example for producing molded textile articles or for the production of threads, fibers and/or filaments.

All melt-spinning methods may be used notably by passing PA11 or the composition of the invention through dies comprising one or more orifices. For producing multifilament threads, mention is made of spinning or spin-drawing or spin-drawing-texturizing methods, whether integrated or not, whatever the spinning speed. It is possible to produce threads by high-velocity spinning, at a speed greater than or equal to 3000 m/min, preferably greater than or equal to 4000 m/min, Such methods are often designated by the following terms: POY (partially oriented yarn), FOY (fully oriented yarn), ISD (integrated spin-drawing), HOY (highly oriented yarn with a speed greater than 5500 m/min). These threads may be texturized according to their intended use. The threads obtained by these methods are suitable quite particularly for producing woven or knitted textile surfaces. According to the invention, the thermoplastic polymeric matrix made of PA11 may be used for producing monofilament threads or monofilaments, multifilament threads or multifilaments, continuous fibers (in reels), and/or discontinuous fibers (cut), Discontinuous PA11 fibers are particularly well suited for mixing with natural fibers.

For individual or monofilament fibers, the counts may extend from 1.5 dtex to 100 dtex/filament, high counts being particularly well suited to industrial applications. Multifilament threads preferably have counts less than or equal to 6 dtex/filament, more preferably less than or equal to 1.5 dtex/filament. For producing fibers, filaments may for example be joined in the form of a mesh or sheet, directly after spinning or in repeated steps, drawn, texturized or crimped and cut. The fibers obtained may be used for producing non-wovens or spun fiber yarns. PA11 or compositions may also be used for the production of flocks. The threads, fibers and/or filaments of the invention may undergo various treatments such as, for example, stretching in a continuous step or in repeated steps, size deposition, oiling, interlacing, texturizing, crimping, stretching, heat treatment for fixing or relaxation, throwing, twisting and/or dying. For dying, mention is made in particular of dying methods in a bath or by jets. Preferred dyes are acidic, metal-containing or non-metal-containing dyes.

The present invention also relates to a textile (or textile article or textile material) obtained at least partially from PA11 as previously defined, being in the form of threads, fibers and/or filaments as previously defined. These textile materials or articles may be cloths or textile surfaces, such as woven, knitted, non-woven or rug surfaces. Such articles may for example be carpets, rugs, furnishings, surface coverings such as coverings intended for sofas, curtains, bed linen, mattresses and pillows, garments and materials.

The textile according to the invention advantageously constitutes a felt, a filter, a film, a gauze, a cloth, a dressing, a layer, a woven fabric, a knitted fabric, an article of clothing, a garment, an article of bedding, an article of upholstery, a curtain, an automobile interior covering, a functional technical textile, a geotextile and/or un agrotextile.

Said textile is advantageously used in the fields of medicine, hygiene, baggage, clothing manufacture, clothing, household equipment and goods, upholstery, carpets, automobiles, industry, notably industrial filtration, agriculture and/or building construction.

The textile according to the invention can notably be used in the fields of use described in the table of page A2.5 of the appendix entitled <<The DRA Textile Products End-Use Consumption Forecasting System” published in 2003 by David Rigby Associates and available at the following internet address: www.davidrigbyassociates.co.uk.

The present invention also relates to textile articles obtained by employing a matrix of PA11 or a thermoplastic composition comprising PA11 according to the invention chosen from the group comprising an extrusion method such as the extrusion of sheets and films, a molding method such as compression molding and an injection method such as injection molding. Films may be obtained in this way by the methods previously mentioned using a flat die. The films obtained may undergo one of various treatment steps such as uniaxial or biaxial stretching, a stabilizing heat treatment, antistatic treatment and/or sizing.

Advantageously, the polyamide 11 content by weight represents at least 50%, preferably at least 80%, of the total weight of the textile according to the invention.

Advantageously, said fibers, said filaments and/or said films based on PA11 have a smooth surface. It has been demonstrated that this increases the bacteriostatic activity of the textile material comprising these fibers and/or filaments.

These textiles according to the invention produced mainly with a PA11 base (comprising at least 50% by weight of PA11) have moreover other advantageous properties. They are light, flexible, soft to the touch, resistant to tearing, cutting, abrasion and pilling, and they appear cold on first contact.

Advantageously, said textile additionally includes natural fibers such as cotton, wool and/or silk, artificial fibers produced from natural raw materials, inorganic fibers such as carbon fibers, glass fibers, silica fibers and/or magnesium fibers, metal fibers and/or synthetic fibers other than PA11.

Advantageously, said textile comprises synthetic fibers obtained from bio-resourced raw materials. Preferably, the textile according to the invention is produced solely from bio-resourced raw materials.

Raw materials of renewable origin or bio-resourced raw materials are understood to mean materials that contain bio-resourced carbon of renewable origin. In point of fact, as against materials coming from fossil material, materials composed of renewable raw materials contain ¹⁴C. The “content of carbon of renewable origin” or the “content of bio-resourced carbon” is determined in application of ASTM standard D 6866 (ASTM D 6866-06) and, as appropriate, ASTM standard D 7026 (ASTM D 7026-04). The first standard describes a test for measuring the ¹⁴C/¹²C ratio of a sample and comparing this with the ¹⁴C/¹²C ratio of a reference sample of 100% bio-resourced origin, to give a relative percentage of bio-resourced C in the sample. The standard is based on the same concepts as for ¹⁴C dating, but without applying dating equations. The ratio calculated in this way is designated as “pMC” (percent Modern Carbon). If the material to be analyzed is a mixture of biomaterial and fossil material (without a radioactive isotope), then the value of pMC obtained is directly correlated to the quantity of biomaterial present in the sample. ASTM standard D 6866-06 proposes several techniques for measuring the ¹⁴C isotope content, based either on LSC (Liquid Scintillation Counting) spectrometry, or on AMS/IRMS (Accelerated Mass Spectrometry coupled with Isotope Radio Mass Spectrometry). The preferred measuring method used in the case of the present invention is mass spectrometry described in ASTM standard D6866-06 (“accelerator mass spectroscopy”).

Textiles of the invention containing polyamide 11 are derived at least partially from bio-resourced raw materials and thus have a bio-resourced carbon content of at least 1%, which corresponds to an isotope ratio of ¹² C/¹⁴ C of at least 1.2×10⁻¹⁴. Preferably, these textiles according to the invention contain at least 50% by weight of bio-resourced carbon based on the total weight of carbon, which corresponds to an isotope ratio ¹²C/¹⁴C of at least 0.6×10⁻¹². This content is advantageously higher, notably up to 100%, which corresponds to a C isotope ratio of 1.2×10⁻¹². The textiles according to the invention may thus contain 100% bio-resourced carbon or, on the contrary, result from a mixture with a fossil origin.

### Example

The example below, which notes the bacterial activity of various textiles, illustrates the present invention without limiting its scope.

A textile produced from a PA11 of the invention is compared with textiles based on other materials normally used in textiles: PA66 (or nylon 66), polyester (PES), cotton, PA6 (or nylon 6), polypropylene (PP), silk, PLA (polylactic acid). PA11 in the example according to the invention has an inherent viscosity substantially equal to 1 (according to the ARKEMA method: 0.5% by weight in metacresol at 20° C., Ubbelohde viscometer).

All measurements of bacterial activity were carried out according to ISO standard 20743 by transfer (XP G 39-010).

PROTOCOL:

Specimen of 38 mm in diameter.

Strains: Staphylococcus aureus

Contact of the specimen for 1 min on agar seeded with an inoculum at 106 UFC/ml, under a weight of 200 g.

Following this contact, the inoculum present on the textile was recovered with the aid of an extraction liquid. This liquid underwent various dilutions which were then themselves able to seed new agars.
Incubation: 24 h at 37° C. in a humid chamber
Measurement of the bacterial population at time 0 h and at 24 h. The count was performed manually.

The results giving bacterial activity are expressed in table 1 below:

Calculation of this bacterial activity corresponds to the difference of UFC number expressed in log between time 24 h and 0 h on the treated product (Δ between −2 and +2):
if this calculation gives a result <0, the activity of the textile is described as bacteriocidal;
if the result is comprised within the range 0 to 2 (including limits), the activity of the textile is described as bacteriostatic;
if this calculation gives a result >2, the textile is classified as neutral or inactive toward bacteria.

TABLE 1
Characterization and measurement of antibacterial activity

| | | Characterization and |
|---|---|---|
| | | measurement of antibacterial |
| | | activity (transfer method) |
| Reference | Composition of the textile | Growth of log (UFC/e) in 24 h |
| Count 1 | PA 66 MM FT 2/78/34 S//Z | 2.87 |
| Count 2 | Polyester FTF 1/167/96 | 2.28 |
| Count 3 | Combed cotton Nm 60/1 | 2.67 |
| Count 4 | BECOOL ® PA6 MM FT 2x84/48 | 2.55 |
| Count 5 | Polypropylene 2/70/50 FTSZ | 2.00 |
| Count 6 | ORGANSIN SILK 8X20/22 | 2.67 |
| Count 7 | Polyester FTF 2/78/47 SZ | 2.79 |
| | COOLMAX ® | |
| Count 8 | Modal ® (cellulose viscose) NM 60/1 | 2.38 |
| Count 9 | 3. PLA (Polylactic acid) FT 167F48 | 2.77 |
| Example | PA11 FT SZ 2/78/30 | 0.37 |
| according to | | |
| the invention | | |

### Observations:

Apart from textiles made of PA11, the other textiles do not exhibit antibacterial activity. PA11 has an antibacterial activity that is the ideal activity sought after.

What is claimed is:

1. A bacteriostatic textile material, comprising polyamide 11 having an inherent viscosity of from 0.5 to 1.7 and wherein said polyamide 11 contains no impurities with a diameter greater than 5 μm.

2. The bacteriostatic textile material of claim 1, wherein the polyamide 11 has an inherent viscosity of 0.5 to 1.5.

3. The bacteriostatic textile material of claim 2, wherein the polyamide 11 has an inherent viscosity of 0.8 and 1.2.

4. The bacteriostatic textile material of claim 1, wherein said material is in the form of a porous membrane, a woven textile or a non-woven textile.

5. The bacteriostatic textile material of claim 1, wherein said material comprises fibers and/or filaments and/or particles based on said polyamide 11.

5. The bacteriostatic textile material as claimed in claim 4, wherein said fibers and/or said filaments have a smooth surface.

6. The bacteriostatic textile material of claim 1, wherein the content by weight of polyamide 11 represents at least 50% of the total weight of the textile.

7. The bacteriostatic textile material of claim 6, wherein the content by weight of polyamide 11 represents at least 80% of the total weight of the textile.

8. The bacteriostatic textile material of claims 1, wherein said polyamide 11 is mixed with at least one filler and/or at least one pigment and/or at least one additive.

9. The bacteriostatic textile material of claim 1, wherein said textile material does not contain any additive that is active toward bacteria or any surface treatment that is active toward bacteria other than polyamide 11.

10. The bacteriostatic textile material of claim 1, wherein said textile material comprises other synthetic fibers obtained from bio-resourced raw materials.

11. The bacteriostatic textile material of claim 1, wherein said textile material further contains natural fibers, artificial fibers produced from natural raw materials, inorganic fibers, metal fibers and/or synthetic fibers.

12. The bacteriostatic textile material of claim 1, consisting solely from bio-resourced raw materials.

13. The bacteriostatic textile material of claim 1, wherein said textile material is selected from the group consisting of a felt, a filter, a film, a gauze, a cloth, a dressing, a layer, a woven fabric, a knitted fabric, an article of clothing, a garment, an article of bedding, an article of upholstery, a curtain, an automobile interior covering, a functional technical textile, a geotextile and an agrotextile.

15. The bacteriostatic textile material of claim 1 comprising an article for use in the fields selected from the group consisting of medicine, hygiene, baggage, clothing manufacture, clothing, household equipment and goods, upholstery, carpets, automobiles, industry, notably industrial filtration, agriculture and building construction.
